(19) United States
(12) Patent Application Publication
(10) Pub. No.: US 2013/0338642 A1
(43) Pub. Date: Dec. 19, 2013

(54) Feeding Tube Clip

(71) Applicant: Randall J. Maulin, Pueblo west, CO (US)

(72) Inventor: Randall J. Maulin, Pueblo west, CO (US)

(21) Appl. No.: 13/213,697

(22) Filed: Aug. 19, 2011

Related U.S. Application Data
(60) Provisional application No. 61/391,757, filed on Oct. 11, 2010.

(51) Int. Cl. A61J 15/00; A61M 39/10

(52) U.S. Cl. CPC A61J 15/0026; A61M 39/1011
USPC 604/533

(57) ABSTRACT

A feeding tube clip has a first tube clasp. The tube clasp has a truncated cone shape with an opening along a length of the truncated cone shape. A frame has a first end and a second end. A clip end connector is attached to the second end of the frame. The clip end connector forms a groove perpendicular to a length of the frame. A clip end has a second tube clasp perpendicular to a mating slot. The mating slot mates to the clip end connector. The mating slot has an opening that aligns with the hole of the clip end connector. In addition a stop clip may be used to engage the clip end. The stop clip has a peg that inserts into the hole of the clip end connector and the aligned opening of the clip end. The stop clip ensures a very strong secure connection.

### RELATED APPLICATIONS

The present invention claims priority on provisional patent application, Ser. No. 61/391,757, filed on Oct., 11, 2010 entitled “Guardian Connection” and is hereby incorporated by reference.

### STATEMENT REGARDING FEDERALLY SPONSORED RESEARCH

Not Applicable

### THE NAMES OF THE PARTIES TO A JOINT RESEARCH AGREEMENT

Not Applicable

REFERENCE TO A SEQUENCE LISTING, A TABLE, OR A COMPUTER PROGRAM LISTING

Not Applicable

### BACKGROUND OF THE INVENTION

Gastrostomy feed tubes are used to help patients receive nutrition who are unable to receive adequate nutrition by eating. Commonly when in use the feeding tube is connected to the feed delivery set. The connection is secured with medical tape. Unfortunately, the tape often comes lose and the result is the contents of the nutritional bag seep out all over the patient and their clothing. This is both uncomfortable, messy and damaging the clothing the patient is wearing.

Thus there exists a need for feeding tube clip that ensures the connection between the feeding tube and the feed delivery set is secure.

### BRIEF SUMMARY OF INVENTION

A feeding tube clip that overcomes these and other problems has a first tube clasp. The tube clasp has a truncated cone shape with an opening along a length of the truncated cone shape. A frame has a first end and a second end, the first end is permanently attached to a portion of a periphery of a wide side of the truncated cone shape. A clip end connector is permanently attached to the second end of the frame. The clip end connector forms a groove perpendicular to a length of the frame. A beveled tip forms one side of the groove. A hole is formed in a center of the clip end connector. A clip end has a second tube clasp perpendicular to a mating slot. The mating slot mates to the clip end connector. The mating slot has an opening that aligns with the hole of the clip end connector. In one embodiment, a stop clip engages the clip end. The stop clip has a peg that inserts into the hole of the clip end connector and the aligned opening of the clip end. The device may be used with or without the stop clip. The stop clip ensures a very strong secure connection.

In operation the user inserts the first tube clasp over the feeding tube end of the connection. The user then attaches the second tube clasp over the feeding tube attached to the feed delivery set. The second tube clasp is slid toward the clip end connector of the frame until the mating slot engages the groove of the clip end connector. Because the groove has a beveled edge on the distal end, the second tube clasp is securely attached to the frame and the device holds the connection between the feeding tube in the patient and feed delivery set. The beveled edge does provide a strain relief mechanism if the patient forgets that they are connected to the feed tube pouch and allows the connection to break away if pulled on too hard. If it is desired that the connection be more secure then the stop clip may be used. The stop clip snap fits over the clip end and clip end connector.

### BRIEF DESCRIPTION OF THE SEVERAL VIEWS OF THE DRAWINGS

FIG. 1 is a cartoon drawing of a feeding tube and feeding bag in accordance with one embodiment of the invention;

FIG. 2 is perspective view of the feeding tube clip and the feeding tube in accordance with one embodiment of the invention;

FIG. 3 is a perspective view of a feeding tube clip in accordance with one embodiment of the invention;

FIG. 4 is a perspective view of a tube holder in accordance with one embodiment of the invention;

FIG. 5 is a side view of a clip end connector in accordance with one embodiment of the invention;

FIG. 6 is a perspective view of a clip end in accordance with one embodiment of the invention;

FIG. 7 is a bottom view of clip end in accordance with one embodiment of the invention;

FIG. 8 is an end view of a stop clip in accordance with one embodiment of the invention; and

FIG. 9 is a side view of a stop clip in accordance with one embodiment of the invention.

### DETAILED DESCRIPTION OF THE INVENTION

The invention is directed to a feeding tube clip has a first tube clasp. The tube clasp has a truncated cone shape with an opening along a length of the truncated cone shape. A frame has a first end and a second end, the first end is permanently attached to a portion of a periphery of a wide side of the truncated cone shape. A clip end connector is permanently attached to the second end of the frame. The clip end connector forms a groove perpendicular to a length of the frame. A beveled tip forms one side of the groove. A hole is formed in a center of the clip end connector. A clip end has a second tube clasp perpendicular to a mating slot. The mating slot mates to the clip end connector. The mating slot has an opening that aligns with the hole of the clip end connector. In one embodiment, a stop clip engages the clip end. The stop clip has a peg that inserts into the hole of the clip end connector and the aligned opening of the clip end. The device may be used with or without the stop clip. The stop clip ensures a very strong secure connection.

In operation the user inserts the first tube clasp over the feeding tube end of the connection. The user then attaches the second tube clasp over the feeding tube attached to the feed deliver set. The second tube clasp is slid toward the clip end connector of the frame until the mating slot engages the groove of the clip end connector. Because the groove has a beveled edge on the distal end, the second tube clasp is securely attached to the frame and the device holds the connection between the feeding tube in the patient and feed delivery set. The beveled edge does provide a strain relief mechanism if the patient forgets that they are connected to the feed tube pouch and allows the connection to break away if pulled on too hard. If it is desired that the connection be more secure then the stop clip may be used. The stop clip snap fits over the clip end and clip end connector.

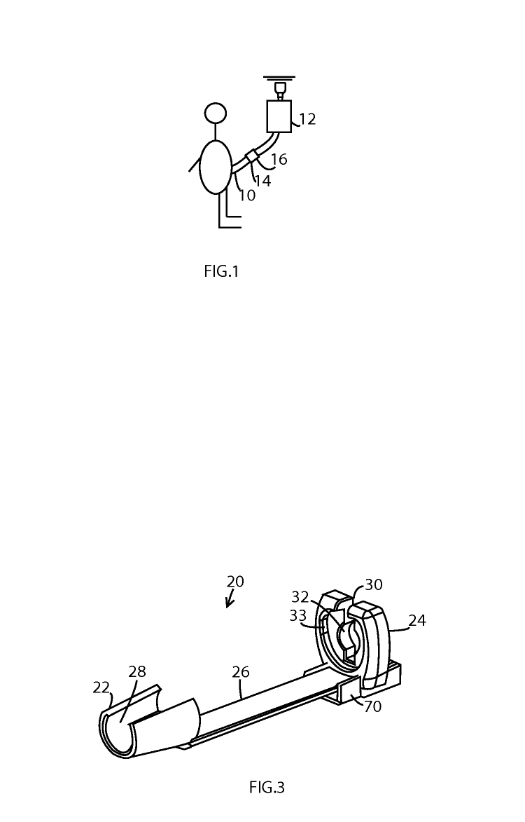

FIG. 1 is a cartoon drawing of a feeding tube 10 and feeding pouch 12 in accordance with one embodiment of the invention. The feeding tube 10 is surgically placed in the person's stomach and has an end 14 that can be connected to a connector 16 on the feeding pouch 12. Commonly, the two tubes are then held together by tape. The invention holds the two tubes together securely without tape.

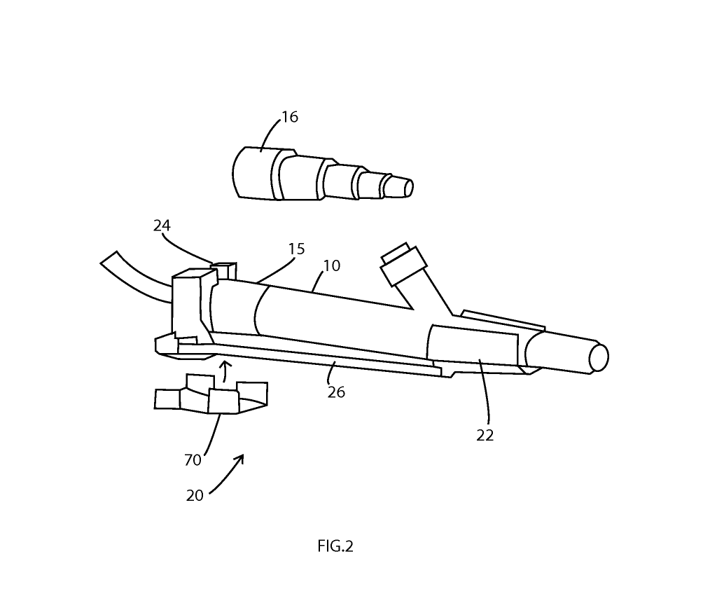

FIG. 2 is perspective view of the feeding tube clip 20 and the feeding tube 10 in accordance with one embodiment of the invention. The feeding tube 10 is inserted into the first tapered tube connector 22. Then the feeding tube 10 is inserted into the feed delivery set connector 15. The connector 15 is placed into the second tube holder 24. If the plug 16 is being used it is inserted into the feed tube 10 and then into the second tube holder 24. If a secure connection is desired the stop clip 70 is inserted into the food clip 20.

FIG. 3 is a perspective view of a feeding tube clip 20 in accordance with one embodiment of the invention. The feeding tube clip 20 has a first tube holder 22 at one end and a second tube holder 24 at the second end connected by a frame 26. The first tube holder 22 is cone shaped and has an opening 28 into which part of the feeding tube is inserted. The second tube holder or guide 24 has a slot 30 for holding a second portion of the feeding tube. The slot 30 has a pair of separated walls with a circular cutout 32 in the center, as best seen in FIG. 5. A cutout ridge 33 in the guide 24 mates with the feed delivery set connector 15 to form a secure connection.

FIG. 4 is a perspective view of a tube holder 40 in accordance with one embodiment of the invention. In one embodiment the feeding tube clip 20 is made of three major parts. The first part is the tube holder 40, which includes the first tube holder 22 and the frame 26. The first tube holder 22 is permanently attached to a first end of the frame 26 on the wide end of the truncated cone shape. The second end of the frame 26 is attached to a clip end connector 42. The clip end connector 42 is permanently attached to the frame 26 and has a hole 44 essentially through its center.

FIG. 5 is a side view of a clip end connector 42 in accordance with one embodiment of the invention. The clip end connector 42 forms a groove 46 perpendicular to the length of the frame 26. One end of the groove 46 is a solid stop 48 and the other end (distal end) of the groove 46 has a beveled tip 50.

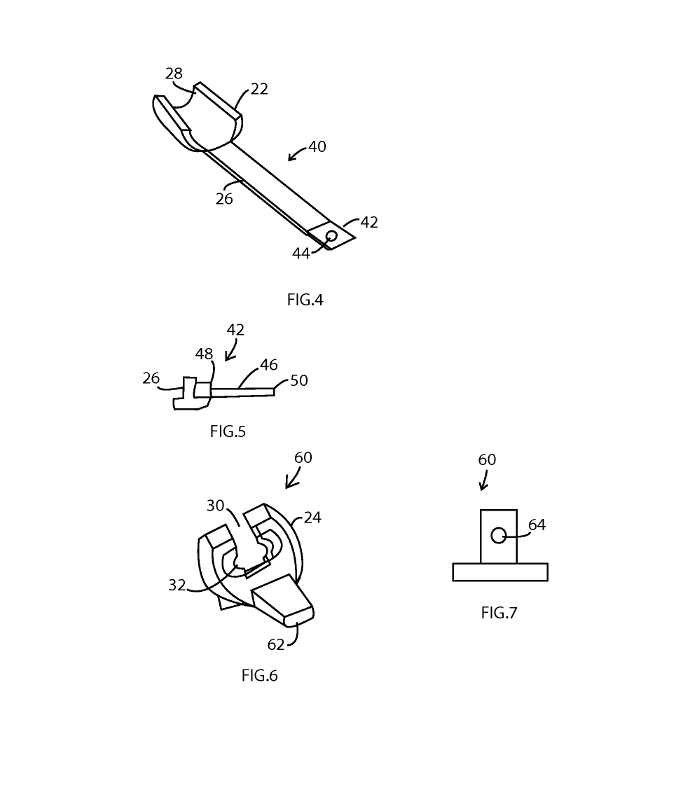

FIG. 6 is a perspective view of a clip end 60 in accordance with one embodiment of the invention. The clip end 60 has a mating slot 62 that is perpendicular to the guide 24. In operation the mating slot 62 of the clip end 60 slides onto the clip end connector 42 and is held in the groove 46 by the beveled tip 50. The beveled tip 50 acts a strain relief mechanism.

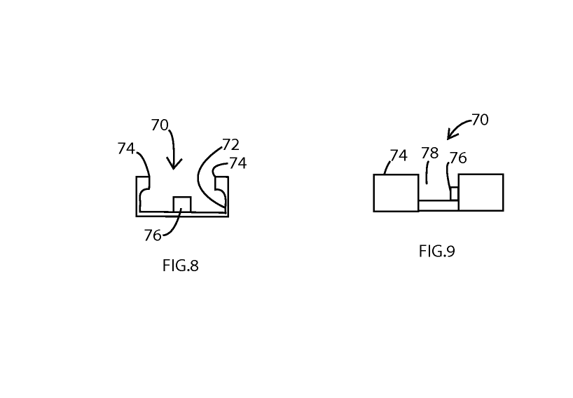

FIG. 7 is a bottom view of clip end 60 in accordance with one embodiment of the invention. This view shows an opening 64 in the bottom of the mating slot 62. The opening 64 aligns with hole 44 of the tube clip connector when the clip end 60 is attached to the tube holder 40.

FIG. 8 is an end view of a stop clip 70 in accordance with one embodiment of the invention. The stop clip 70 has a channel 72 with a pair of overlapping lips 74. A peg 76 extends into the channel 72.

FIG. 9 is a side view of a stop clip 70 in accordance with one embodiment of the invention. This view shows a cutout 78. This cutout 78 mates with walls of the guide 24. The overlapping lips 74 engage the top of the mating slot 62 of the clip end 60. The peg 76 is inserted into the opening 64 in the mating slot of the clip end 60 and the aligned hole 44 in the clip end connector 42. This prevents the two tube holders 24, 28 from becoming detached. Note that the device may be used with or without the stop clip 70.

In operation the user inserts the first tube clasp over the feeding tube end of the connection. The user then attaches the second tube clasp over the feed delivery set connector. The second tube clasp is slid toward the clip end connector of the frame until the mating slot engages the groove of the clip end connector. Because the groove has beveled edge on the distal end, the second tube clasp is securely attached to the frame and the device holds the connection between the feeding tube in the patient and the feed deliver set. The beveled edge does provide a strain relief mechanism if the patient forgets that they are connected to the feed tube pouch and allows the connection to break away if pulled on too hard. If it is desired that the connection be more secure then the stop clip may be used. The stop clip snap fits over the clip end and clip end connector.

While the invention has been described in conjunction with specific embodiments thereof, it is evident that many alterations, modifications, and variations will be apparent to those skilled in the art in light of the foregoing description. Accordingly, it is intended to embrace all such alterations, modifications, and variations in the appended claims.

What is claimed is:

1. A feeding tube clip, comprising:
a tube holder having a first end with a cone shaped clip and a second end with a clip end connector; and
a clip end having a guide perpendicular to a mating slot that mates to the clip end connector of the tube holder.

2. The feeding tube clip of claim 1, further including a stop clip that engages the clip end.

3. The feeding tube clip of claim 2, wherein the stop clip includes a peg that mates with an opening of the clip end and a hole in the clip end connector.

4. The feeding tube clip of claim 1, wherein the clip end of the tube holder has beveled tip.

5. The feeding tube clip of claim 1, wherein the guide has a pair of separated walls that have a circular cutout near the middle of the pair of separated walls.

6. A feeding tube clip, comprising:
a first tube holder;
a second tube holder; and
a frame connecting the first tube clip to the second tube clip.

7. The feeding tube clip of claim 6, wherein the frame has a clip end that engages a mating slot of the second tube holder.

8. The feeding tube clip of claim 6, wherein the first tube holder is permanently attached to the frame.

9. The feeding tube clip of claim 7, wherein the clip end of the frame has beveled tip.

10. The feeding tube clip of claim 7, further including a stop clip that engages the clip end.

11. The feeding tube clip of claim 6, wherein the first tube holder has a cone shape with an opening.

12. The feeding tube clip of claim 6, wherein the second tube holder has a strain relief mechanism.

13. The feeding tube clip of claim 6, wherein the frame includes a strain relief mechanism.

14. A feeding tube clip, comprising:
a first tube clasp, having a truncated cone shape with an opening along a length of the truncated cone shape;
a frame having a first end and a second end, the first end permanently attached to a portion of a periphery of a wide side of the truncated cone shape;
a clip end connector permanently attached to the second end of the frame, the clip end connector forming a groove perpendicular to a length of the frame, a beveled tip forming one side of the groove, further including a hole in a center of the clip end connector; and
a clip end having a second tube clasp perpendicular to a mating slot, the mating slot mates to the clip end connector, the mating slot have an opening that aligns with the hole of the clip end connector.

15. The feeding tube clip of claim 14, further including a stop clip that engages the clip end.
